(19) United States
(12) Patent Application Publication
(10) Pub. No.: US 2009/0204100 A1
(43) Pub. Date: Aug. 13, 2009

(54) BODY COVER AND A METHOD OF COMMUNICATING A VARIATION IN TEMPERATURE OF THE SKIN

(75) Inventors: Liesbeth Van Pieterson, Eindhoven (NL); Margreet De Kok, Eindhoven (NL); Sima Asvadi, Eindhoven (NL)

Correspondence Address: PHILIPS INTELLECTUAL PROPERTY & STANDARDS, P.O. BOX 3001, BRIARCLIFF MANOR, NY, 10510, US

(73) Assignee: Koninklijke Phillips electronics N.V., Eindhoven (NL)

(21) Appl. No.: 12/304,152

(22) PCT Filed: May 9, 2007
(86) PCT No.: PCT/IB2007/051745

§ 371 Date: Dec. 10, 2008

(30) Foreign Application Priority Data
Jun. 12, 2006 (EP) 06115285.6

(51) Int. Cl. A61B 5/01; A61F 13/00; A61N 5/06; A61M 31/00

(52) U.S. Cl. 604/503; 600/549; 604/305; 607/88

(57) ABSTRACT

Body cover for application to the skin, comprising at least one temperature sensing element, wherein the body cover is substantially flexible, wherein the at least one temperature sensing element, when applied to the skin, is configured to sense the temperature of proximate skin at least locally with respect to the body cover and to convert the locally sensed temperature into a visual and/or electrical signal.

The invention relates to a body cover for application to the skin and a method of communicating a variation in temperature of the skin.

For example in hospitals, wound inspection is done on a regular basis to prevent severe inflammation. Problems with wound healing are encountered with many patients after surgery. In most cases these patients are allowed to go home, but have to return to the hospital for inspection of the wounds. During inspection attention may be paid to size, color, smell and temperature of the wound. In case of infection, the temperature of the skin surrounding the wound may increase by several degrees Celsius. Normally, the external skin temperature is approximately 32° C. and this can increase to approximately 37° C. in case of an infection.

Another clinical situation in which early detection of infection is important is in case of infusion to administer drugs and/or food intravenously or catheters to allow body fluids to leave the patient. The infusions or catheters are often a source of bacteria and will lead to infection of the arteries and underlying tissue, which may slow down recovery of the patient. Also when regularly pumping insulin, there is a high risk of infection.

In many cases the possible infection sites are hidden under clothes, plasters and/or bandages and may be invisible to or overlooked by the patients. Furthermore, replacing a wound dressing can be painful for the patient and induce further contamination.

A goal of the invention is to enhance convenience for the patient, at least near a skin location.

This goal and other goals may be achieved by a body cover for application to the skin, comprising at least one temperature sensing element, wherein the body cover is at least partly flexible, wherein the at least one temperature sensing element, when applied to the skin, is configured to sense the temperature of proximate skin at least locally with respect to the body cover and to convert the locally sensed temperature into a visual and/or electrical signal.

The above-mentioned and other goals may also be achieved by a method of communicating a variation in temperature of the skin, wherein a substantially flexible cover is applied to the skin, wherein local variations in temperature of the part of the skin that is covered by the cover are sensed by the cover and wherein signals that correspond with said local variations are signaled.

In clarification of the invention, embodiments thereof will be further elucidated with reference to the drawing. In the drawing:

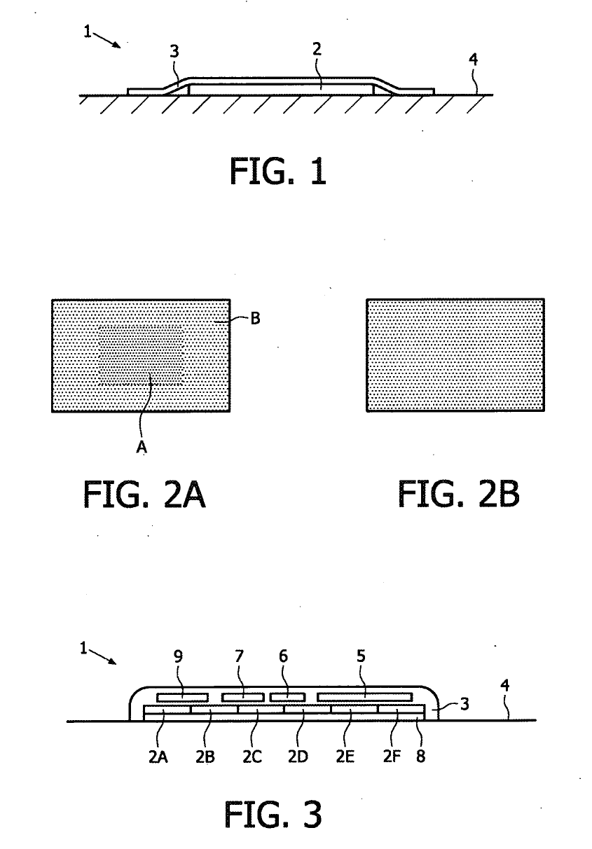

FIG. 1 shows a section of an embodiment of a body cover;

FIGS. 2A and B show top views of an embodiment of a body cover;

FIG. 3 shows a section of an embodiment of a body cover;

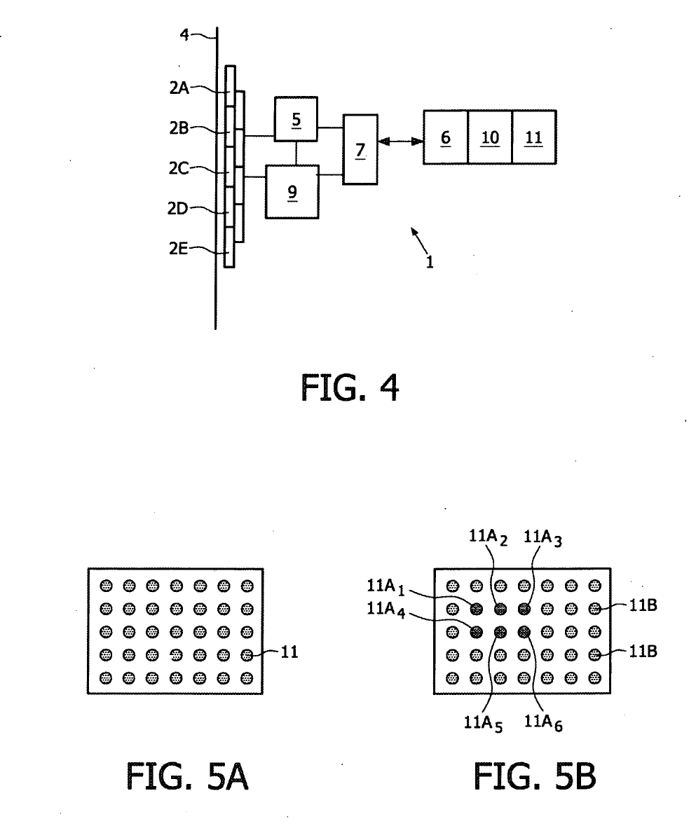

FIG. 4 shows a diagram of an embodiment of a body cover;

FIGS. 5A and B show top views of an embodiment of a body cover;

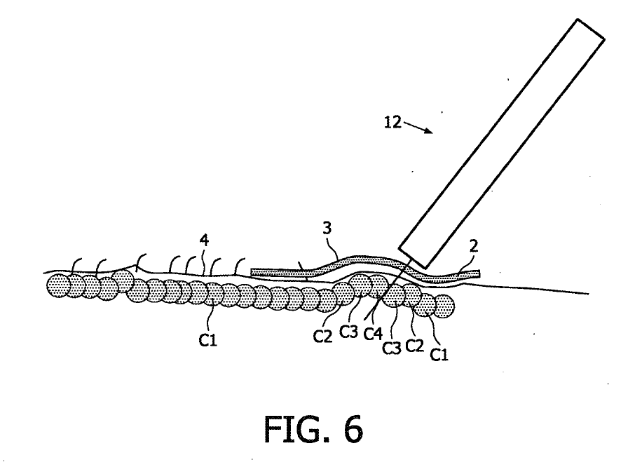

FIG. 6 shows an embodiment of a combination of a body cover and an administering device.

In this description, identical or corresponding parts have identical or corresponding reference numerals. The exemplary embodiments shown should not be construed to be limiting in any manner and serve merely as illustration.

In FIG. 1, a section of an embodiment of a body cover 1 is shown. Here, body cover 1 comprises a temperature sensing element 2 that is applied to the skin 4 of a patient with the aid of a bandage 3. The body cover 1 itself may also function as a wound dressing and/or bandage 3. The temperature sensing element 2 senses the temperature of the skin 4 that is near the temperature sensing element 2, wherein ‘skin’ has to be explained as comprising at least the body exterior, i.e. also open wounds. In the embodiment shown in FIG. 1, the temperature sensing element 2 comprises thermochromic material, for example thermochromic dyes, paint, ink, paper, liquid crystalline material etc. A choice between thermochromic materials may for example depend on the desired level of accuracy or the desired range of temperatures that can be sensed and displayed.

The bandage 3 can for example be at least partly transparent, such that color changes of the temperature sensing element 2 can be observed from outside the bandage 3. Of course, the temperature sensing element 2 may itself comprise a bandage 3 and be at least partly or fully flexible.

Near a wound, or when an inflammation occurs the temperature is different from the temperature of the surrounding skin 4. Therefore, in an advantageous embodiment the temperature sensing element 2 is configured to locally assume a color corresponding with a local variation in temperature of the nearby skin 4. For example in case of a wound and/or inflammation (relatively high temperature), the temperature sensing element 2 locally shows a variation in color at the site of the wound and/or inflammation, different from its color near the surrounding skin 4 (relatively low temperature). Hence, a color gradient is presented by the temperature sensing element 2 that approximately corresponds with the varying temperature of the underlying skin 4. This is shown in FIG. 2A. As shown in the figure, an area A has a substantially darker color than an area B, wherein the color of the area A corresponds with an approximate temperature of 37° C., corresponding with the temperature of an inflamed wound, and the color of the area B corresponds with an approximate temperature of 32° C., corresponding with the temperature of the skin 4 in normal conditions, more or less. Of course said temperatures might vary under specific conditions and per person. From area B to area A the temperature of the underlying skin 4 gradually rises such that a color gradient is presented. In this way, without removing the body cover, the state of the wound can be observed, wherein the local color variations in the body cover 1 present local states of the wound. A color that is presented in the area A by the body cover 1 and corresponds with a certain high value may imply a warning for the patient, since the local temperature shift might be caused by infection. FIG. 2B shows a state of the body cover 1 wherein the temperature of the skin 4 underlying the body cover 1 is substantially the same, wherein little or no color variation can be observed.

The temperature of the skin 4 is also influenced by factors such as physical movement and/or outside temperature. Generally, the change in temperature will then not confine to very local temperature changes, but over larger surfaces of the body. If this occurs, in an embodiment, the whole temperature sensing element 2 shows a color shift, so that the relative differences in local color of the temperature sensing element 2 near for example an infected part near a healthy part will be maintained. In this way, temperature changes over the whole body or larger surfaces thereof are compensated and relative temperature differences between for example infected and healthy skin parts are represented by the invention.

In another embodiment, as shown in FIG. 3 in cross section or FIG. 4 in a diagram, a temperature sensing element 2 comprises one or more temperature sensing silicon chips 2 and/or thermocouples. As known in the art, these temperature sensing elements 2 are capable of varying an electrical signal when the temperature of at least a part of the temperature sensing element 2 changes. These temperature sensing elements 2 are known for example from printed circuits and digital thermometers. In FIG. 3, an embodiment is shown comprising a body cover 1 provided with temperature sensing elements 2A-F, which may for example be silicon chips or thermocouples. Furthermore, a protective cover 3, a storage arrangement 6 for storing at least sensed periodic temperature changes, a preferably wireless communication circuit 7 for communicating information to a distant computer and/or receiving settings, and/or a power supply 5 for supplying power to at least one of the elements 2, 6, 7 may be provided. Of course, instead of the wireless communication circuit 7, the body cover 1 can also be wired to a distant computer. A transfer layer 8 may for example be provided that protects the skin 4 from the temperature sensing elements 2 and transfers skin temperature to the temperature sensing elements 2. This transfer layer 8 may also conveniently comprise a disinfection layer and/or any kind of medication and/or gel, for example configured to recover and/or protect the skin 4.

The protective cover 3 may for example include a local display, for example comprising LED or LCD techniques, for converting electrical signals that are output by the temperature sensing elements 2 into a visual signal, such that the wearer of the body cover 1 or another person or a visual inspection aid such as a camera, optical sensor or the like, can read the state of the skin 4 from the body cover 1. A processing circuit 9 can be incorporated in the body cover 1 to process certain information locally. In an embodiment, the protective cover 3 comprises a housing for the electrical elements 2, 5, 6, 7, 9 of the body cover 1 and is at least partly flexible. The wireless communication circuit 7 can directly transmit received signals to a distant and/or or mobile communication device 10 such as a personal computer, a server, a database, PDA, telephone, etc. that may have a display 11, or other means of communication, for example audible communication means such as a loudspeaker. The local temperature of the skin 4 is measured by means of the temperature sensing elements 2 and is displayed on said distant or local communication means such as a display 11. For example, the display 11 may show an approximate representation of a wound underlying the body cover 1 on the base of the measured signals.

The signals that are sent by the temperature sensing elements 2 are stored in a storage arrangement 6, e.g. a memory chip, so that periodic temperature variations can be stored with time. This may give an indication of the healing process. The storage arrangement 6 can be incorporated into the body cover 1 such that it stores information locally, but also a distant storage arrangement 6 may be used, for example in said distant computer and/or on any kind of suitable data carrier.

Again, when a rise or fall in temperature over a relatively large body part occurs, for example during normal physical activity or a change in outside environmental temperature, a temperature shift might occur, which can then be leveled out. Also it may be determined if a temperature shift is relatively temporal or relatively constant, so that it can be determined whether an adjustment of a treatment is necessary. For example, if a periodic temperature shift at the site of a wound corresponds to a temperature shift in the same period elsewhere on the skin 4, this might be related to general body temperature shifts that have occurred, wherein no interaction may be needed. Also small variations in temperature might occur in short time periods that may be relatively harmless. A computer or a person may base the decision if interaction is needed upon predetermined data, for example comprising specific periods, values or other factors.

Preferably, the body cover 1 is substantially flexible. The body cover 1 may for example comprise textile with integrated temperature elements 2 or fragments of relatively hard material that can be moved with respect to each other. Also the body cover 1 may be made of relatively bendable material such that it can be deformed by hand and may approximately be brought into conformity with the skin form of a patient. In particular embodiments the temperature sensing elements 2 are integrated with clothing, thin film material, wound dressings (plasters, bandages) or the like. The temperature sensing element 2, particularly when it is made of thermochromic material such as liquid crystal material, can be coated or impregnated on material such as yarns or fabric. The liquid crystal material may also be deposited on thin films, which can be integrated with textiles, for example in the form of patches. Thermochromic dyes, pigments and inks can be printed on textile materials to form specific patterns by processes such as screen printing or ink jet printing. Also other temperature sensing elements 2 that are connected to a circuit, for example silicon chips and/or thermocouples, can be incorporated into or applied to wearable materials such as textiles. For example, an array of electrodes that are connected to the array of temperature sensing elements 2 may be woven and/or embroidered with the yarns. As is described in the above, a comfortable and wearable and wireless flexible body cover 1 that signals inflammation of the wound in an early stage can be achieved, whereas also in other ways flexible body covers 1 according to the invention can be achieved.

FIGS. 5A and B are schematic top views of a body cover 1 for example comprising silicon chips or thermocouples as temperature sensing elements 2. For the sake of clarity, displaying means, such as pixels and/or spots 11 each correspond with a temperature sensing element 2, of course in practice much higher resolutions can be achieved and the ratio of pixels against temperature sensing elements 2 may vary. FIG. 4A shows an embodiment wherein the temperature of the underlying skin has no or little variation. In FIG. 4B the underlying skin 4 has a locally varying temperature, which is represented by a color gradient in the form of pixels 11A₁-11A₆. For example, the pixel 11A₂ may represent a skin temperature of approximately 37° C., pixels 11A₁, 11A₃-11A₆ a skin temperature of approximately 35° C. and pixels 11B a skin temperature of approximately 32° C.

In particular embodiments, the body cover 1 is integrated or otherwise combined with other sensing elements that sense particular characteristics of the skin 4, for example color sensors that measure the reflectivity and/or emissivity of the skin 4 and/or humidity sensors that measure the humidity near the skin. Also the scent of a wound can be measured. A combination of multiple sensing mechanisms may upgrade the quality of the determination of the state of the skin 4 and hence enhance early warning and/or recovery.

Furthermore, the body cover 1 may advantageously be combined with administering arrangements, for example arrangements for administering light and/or arrangements that administer for example fluids or creams for the treatment of wounds. The invention may for example be integrated with flexible light sources for the administration of phototherapy, including (organic) light emitting diodes, such that a flexible self-monitoring and self-administering system is obtained. In other words, the body cover 1 may be configured such that a sensed local temperature variation triggers the local administration of for example light or medication such that healing may be expedited, whereas physical mobility is largely maintained.

In another embodiment, the body cover 1 is connected with an administering device 12 for administering fluids to the human body, such as catheters and/or infusions, as shown in FIG. 6. An example of this is the administration of insulin. With these types of penetrating administering devices 12, there is a relatively high risk of inflammation. The body cover 1 may be combined with a type of sticking bandage 3, for example of the type that is commercially known as “Tegaderm®”, manufactured by 3M. Because the bandage 3 is lying across the site of penetration of the infusion, infections might be overlooked. A temperature sensing element 2 can be incorporated into said bandage 3. As shown in FIG. 6, temperature spots C₁-C₄ indicate a gradually rising temperature at the site of infusion, wherein spots C₁ for example indicate a temperature of approximately 32° C., C₂ of approximately 33° C., C₃ of approximately 34° C. and C₄ of approximately 35° C. At C₄, the temperature has to be carefully monitored for further inflammation.

In general, the body cover 1 will send signals to the patient or aid that in specific cases warn about the state of the infection site. For example, a silicon chip senses a local extreme skin temperature which is processed by the processing circuit 9 and compared with predetermined values, which are at least partly stored in the storage arrangement 6. Information can be output to a mobile device 10, which warns the patient or a doctor via display 11.

Temperature sensing elements 2 and/or displays 11 may show colors that may comprise any color and/or shade of color. Differences in color may also comprise differences in reflectivity and/or intensity. Other visual signals may also be presented in any form, for example by means of characters, but also a representation of the skin 4 underlying the body cover 1 can be rendered.

In an embodiment of the body cover 1 that comprises thermochromic materials, the resulting color shift in these materials can be converted into electrical signals, for example by means of color sensors. In this way, the state of the skin 4 can be communicated and/or stored for example to/on a computer and is not limited to communication to persons that are near the body cover 1, using thermochromic materials.

It will be obvious that the invention is not limited in any way to the exemplary embodiments that are represented in the description and the drawings. Many variations and combinations are possible within the framework of the invention as outlined by the claims. A body cover 1 may for example be used for any type of temperature sensing, not just for wound healing as mentioned above, but also for example for cosmetic purposes. Combinations of one or more aspects of the embodiments or combinations of different embodiments are possible within the framework of the invention. All comparable variations are understood to fall within the framework of the invention as outlined by the claims.

1. Body cover for application to the skin, comprising at least one temperature sensing element, wherein the body cover is at least partly flexible, wherein the at least one temperature sensing element, when applied to the skin, is configured to sense the temperature of proximate skin at least locally with respect to the body cover and to convert the locally sensed temperature into a visual and/or electrical signal.

2. Body cover according to claim 1, wherein said visual signal represents a color variation in the temperature sensing element, preferably a gradient of at least one color that corresponds with said locally sensed temperature.

3. Body cover according to claim 1, wherein the body cover comprises power supply connecting means, a processing circuit configured to process said electrical signals and a storage arrangement, or at least connecting means for the storage arrangement.

4. Body cover according to claim 1, wherein the body cover comprises clothing, a plaster and/or bandage.

5. Body cover according to claim 1, wherein the body cover is at least partly disinfectious.

6. Body cover according to claim 1, wherein the body cover is configured to be penetrated and/or connected with an administering device, for example a needle and/or catheter.

7. Body cover according to claim 1, wherein the at least one temperature sensing element comprises a silicon chip and/or thermocouple.

8. Body cover according to claim 1, wherein the body cover comprises a user interface.

9. Body cover according to claim 1, wherein the at least one temperature sensing element comprises a thermochromic and/or liquid crystalline material.

10. Body cover according to claim 1, wherein the cover comprises a color reading circuit to convert visual signals into electrical signals.

11. Body cover according to claim 1, wherein the body cover comprises a wireless communication circuit to transmit said electrical signals to a processing unit such as a computer or display unit.

12. Body cover according to claim 1, wherein the at least one temperature sensing element is woven, coated, impregnated and/or embroidered in and/or to the body cover.

13. Body cover according to claim 1, wherein the body cover comprises an administering arrangement, for example at least one light source for phototherapy and/or a disinfection fluid administering arrangement.

14. Body cover according to claim 1, wherein the body cover comprises other sensing elements for near the skin such as color sensing and/or fluid sensing elements.

15. Method of communicating a variation in temperature of the skin, wherein a substantially flexible cover is applied to the skin, wherein local variations in temperature of the part of the skin that is covered by the cover are sensed by the cover and wherein signals that correspond with said local variations are signaled.

16. Method according to claim 15, wherein periodic variations that are sensed over respective time periods are processed and/or stored in a storage arrangement and wherein periodic variations of a local part of the skin are compared with periodic variations over at least another part of the skin than said local part of the skin.

17. Method according to claim 16, wherein when said periodic variation of said local part of the skin according to said preset values corresponds with periodic variations of other parts of the skin over substantially the same period(s), the variations are leveled out.

18. Method according to claim 15, wherein said local and/or periodic variations correspond with a variation in and/or a gradient of one or more colors.

19. Method according to claim 15, wherein a sensed signal triggers automatic administration for healing
